(19) United States
(12) Patent Application Publication
(10) Pub. No.: US 2022/0157415 A1
(43) Pub. Date: May 19, 2022

(54) SYSTEM AND METHOD FOR EXAMINING TEST SAMPLES ON A VIRTUAL PLATFORM

(71) Applicants: Kawaldeep Arora, New Delhi (IN); Ankur Singh Bist, New Delhi (IN); Saksham Chaurasia, Gorakhpur (IN); Roshan Prakash, Udupi (IN); Vikas Pandey, New Delhi (IN); Suraz Kottakki, Danang (VN); Sharada Dutt Yadav, New Delhi (IN)

(72) Inventors: Kawaldeep Arora, New Delhi (IN); Ankur Singh Bist, New Delhi (IN); Saksham Chaurasia, Gorakhpur (IN); Roshan Prakash, Udupi (IN); Vikas Pandey, New Delhi (IN); Suraz Kottakki, Danang (VN); Sharada Dutt Yadav, New Delhi (IN)

(73) Assignee: EONBT Private Limited, Kanpur (IN)

(21) Appl. No.: 17/207,853

(22) Filed: Mar. 22, 2021

(30) Foreign Application Priority Data
Nov. 16, 2020 (IN) 202011049830

(51) Int. Cl. G16H 10/40; G06K 9/00; H04L 9/32; G06N 20/00; G16H 10/60

(52) U.S. Cl. CPC G16H 10/40; G06K 9/00275; H04L 9/3231; G06N 20/00; G06K 2209/01; H04L 9/3263; G16H 10/60; H04L 2209/38; G06K 9/00456

(57) ABSTRACT

Disclosed is a system for authentication and examination of test samples (200) including a kit authentication module (220), a testing kit (210), an identity authentication module (230), an examination module (240), and a medical certificate module (250). Further is disclosed a method for authentication and examination of test samples (100) utilising the above mentioned components of the system for authentication and examination of test samples (200) along with a user module (260).

### FIELD OF INVENTION

The present embodiment generally relates to analysis of test samples from a plethora of testing medical equipment. More particularly, the present invention relates to a system and a method for authentication and examination of test samples in an automated manner utilising artificial intelligence and blockchain technologies.

### BACKGROUND OF THE INVENTION

This section is intended only to provide background information pertaining to the similar field of the present invention, and may be used only to enhance the understanding of the present invention and not as admissions of prior art.

In the recent years, there has been a gradual growth in the field of rapid diagnostics, which has saved many a human lives from peril by quickly diagnosing a condition at hand. Such tests are generally performed with a few drops of human serum, plasma, or blood, depending on the kind of condition to be diagnosed.

Conventionally, rapid diagnostics may relate to rapid test means having qualitative or semi-quantitative in-vitro diagnostic medical devices, used singly or in a small series, which involve non-automated procedures and have been designed to provide a quick result. A rapid diagnostic test relates to a medical diagnostic test that is quick and easy to perform. Suitable for a preliminary or emergency medical screening, they can also be used in medical resources with limited resources.

Rapid diagnostic tests further allow point-of-care testing in primary care for conditions that formerly only laboratory tests could measure—providing same-day results within two hours, and in some cases, even within 20 minutes.

Over the years, various complex laboratory tests have been converted into rapid diagnostic tests for a plethora of conditions and these tests aid humankind in a living a healthy lifestyle by on-time detection and appropriate care of a particular, or even at times, more than one conditions.

However, often such rapid testing kits may display false-positive or false-negative results. Additionally, there is no way to authenticate rapid testing kits, which leads to inflation of duplicate testing kits in the market, which may provide false-positive or false-negative diagnoses, causing conditions to either go undetected, or for people to waste a large sum of money in additional medical scans for treatment of conditions that do not exist.

Additionally, in order for a person having a condition as detected by a rapid test kit to avail a medical certificate, the person has to spend additional economies to see a medical professional which issues the medical certificate. This involves a great deal of time being spent by general public and medical examiners on sure-set cases already determined by the rapid test kit.

Therefore, in light of the above-mentioned limitations of the existing rapid testing kits, systems and methods thereof; there exists a need of a system and method for authentication and examination of test samples. More particularly, these exists a need to eliminate traditional systems and methods of rapid testing kits and introduce a rapid testing kit with an encoding placed on the body on the testing kit, a distributed ledger of information which assists in authenticating the testing kit, an artificial intelligence module which examines the testing kit once a test has been performed and provides a diagnoses, and another distributed ledger of information which provides medical certificates based on the diagnoses.

### SUMMARY OF INVENTION

In light of the limitations of the existing conventional systems as discussed above, it is evident that there arises a need of a system and method for authentication and examination of test samples along with a rapid testing kit thereof, having an encoding placed on the body on the testing kit; a distributed ledger of information which assists in authenticating the testing kit, an artificial intelligence module which examines the testing kit once a test has been performed and provides a diagnoses, and another distributed ledger of information which provides medical certificates based on the diagnoses for substantially overcoming the above-mentioned limitations.

The present disclosure portrays a testing kit (210), a kit authentication module (220), an identity authentication module (230), an examination module (240), a medical certificate module (250) and a user module (260), making it a time efficient and appropriate process for ensuring distribution of authenticated test kits, so that tests are being performed by authenticated persons, an appropriate and time-efficient analysis of performed tests and an appropriate distribution of medical certificates wherever applicable.

Another object of the present disclosure is to incorporate a unique identity code onto every test kit so as to ensure distribution of only authentic test kits, thereby reducing the inflation of duplicate test kits which might deliver inappropriate results in the market. Yet another object of the present disclosure is to incorporate a distributed ledger of information which authenticates the test kit by revisiting unique code information being generated while manufacture of the test kits, and verifying the authenticity of the test kits.

Another object of the present invention is to provide an identity authentication module utilising distributed ledger technology which allows the input of identity documents and utilises optical character recognition in order to understand the same, and verify a person's identity from governmental databases. Yet another object of the present invention is to provide the identity authentication module to have a face match and a liveliness test in order to ensure that the identity of the person using or performing the test using the test kit is appropriate and reduce the wrongful usage of the test kits.

Yet another object of the present invention is to provide an examination module facilitated with deep learning, and artificial information compatibility including image processing so as to appropriately capture the results of a test kit, and provide a diagnoses from the test kit. Another object of the present invention is to also predict the intensity of a condition based on the performed result on the test kit.

Another object of the present invention is to integrate distributed ledger technology or blockchain technology in a medical certificate module, which dispenses medical certificates based on the diagnoses achieved by the result of the test kit, for a person or user. This assists in reducing the time and resources spent on availing a medical certificate from a medical practitioner separately.

Yet another object of the present invention is to integrate all above disclosed modules and technologies into the system and method for authentication and examination of test samples in order to ensure that authenticates test kits are being distributed, appropriate personnel are performing the tests, appropriate examination of the test kits is being performed, and medical certificates are being dispensed appropriately to the respective personnel.

In an aspect, embodiments of the present disclosure provide a system for authentication and examination of test samples (200) including a kit authentication module (220), a testing kit (210), an identity authentication module (230), an examination module (240), and a medical certificate module (250).

As used herein, the testing kit (210) is the physical kit which is facilitated to provide test results once a sample is provided to it. Further, a user connects with the system for authentication and examination of test samples (200) via a user module (260). The user module (260) is communicable coupled with the kit authentication module (220), the identity authentication module (230), the examination module (240), and the medical certificate module (250).

### BRIEF DESCRIPTION OF THE DRAWINGS

Other objects, features, and advantages of the embodiment will be apparent from the following description when read with reference to the accompanying drawings. In the drawings, wherein like reference numerals denote corresponding parts throughout the several views:

FIG. 1 illustrates a process flow diagram depicting a method for authentication and examination of test samples (100), in accordance with an embodiment of the present disclosure.

FIG. 2 illustrates a block diagram depicting a system for authentication and examination of test samples (200), in accordance with an embodiment of the present disclosure.

FIG. 3 illustrates a process flow diagram depicting a method for inputting of information (300) in the system for authentication and examination of test samples (200), in accordance with an embodiment of the present disclosure.

FIG. 4 illustrates a process flow diagram depicting a method for authentication of test kits (400) by the system for authentication and examination of test samples (200), in accordance with an embodiment of the present disclosure.

FIG. 5 illustrates a process flow diagram depicting a method for registration of test kits (500) by the system for authentication and examination of test samples (200), in accordance with an embodiment of the present disclosure.

FIG. 6 illustrates a process flow diagram depicting a method for authentication and examination of test samples (600), in accordance with an embodiment of the present disclosure.

### DETAILED DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Throughout the present disclosure the term “a system for authentication and examination of test samples (200)” refers to a system which provides authentication of a testing kit, authentication of a user's identity, examining of test results and providing a diagnosis, and dispensing medical certificates to the user on the basis of the diagnosis. In light of the present disclosure, the system for authentication and examination of test samples (200) may be utilised for over-the-counter testing kits like in-vitro testing kits, and also to other medical devices which require a diagnosis to be attained after a test is performed.

Furthermore, the system for authentication and examination of test samples (200) includes a kit authentication module (220), a testing kit (210), an identity authentication module (230), an examination module (240), and a medical certificate module (250); and is utilised by a user via a user module (260) which is communicably coupled to the system for authentication and examination of test samples (200).

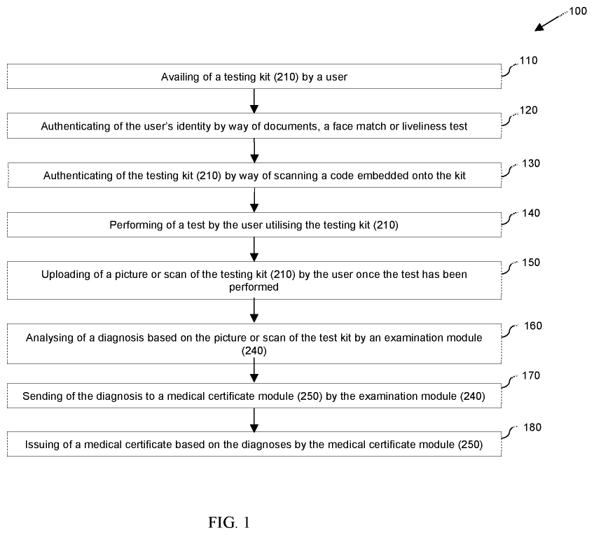

Referring to FIG. 1 illustrates process flow diagram depicting a method for authentication and examination of test samples (100), in accordance with an embodiment of the present disclosure. Furthermore, the method for authentication and examination of test samples (100) comprises of multiple steps as recited below.

At step 110, a user avails a testing kit (210). The user may avail the testing kit (210) from a chemist, a medical store, a medical retailer, a medical manufacturer, or an online retailer. When a user is prepared to perform a test using the testing kit (210), at step 102, the user's identity is authenticated using a user module (260), which is connected to an identity authentication module (230).

The authentication of a user's identity may be done by a plurality of identity documents, a face match or a liveliness test. The user may upload documents onto a user interface (265) in the user module (260). Since the user module (260) is connected to the identity authentication module (230), the documents are sent to the identity authentication module (230). The identity authentication module (230) comprises of an identity module (336), an identity database (234), and an optical character recognition module (235). Further, the identity module (336) comprises of a document module (231), a face match test (232), and a liveliness test (233).

Once the documents are provided to the identity authentication module (230), they are understood by the document module (231) which assists the optical character recognition module (235) to understand the documents and extract identity information. Thereon, the extracted identity information is cross-verified with the identity database (234).

The identity database (234) may pertain to a unique ledger of identities provided by a system for authentication and examination of test samples (200), or it may even be a ledger of identity information availed from governmental portals like the NHS.

Further, at step 130, the testing kit (210) is authenticated by the user using a kit authentication module (220). The testing kit (210) has a kit body (211) provided with a unique code (212). Furthermore, the kit authentication module (220) comprises of a code reader (221), and a ledger of encoded information (222). The unique code (212) placed on the kit body (211) is read using the code reader (221), and the perceived information is thereon verified with the ledger of encoded information (222).

At step 140, the user performs the test in the prescribed manner depending on which test is being performed. As stated above, the system for authentication and examination of test samples (200) may be utilised for over-the-counter testing kits like in-vitro testing kits, and also to other medical devices which require a diagnosis to be attained after a test is performed.

The testing kit (210) further comprises a sample storage area (213), and a test card (214). The sample storage area (213) is utilised for storing a sample, preferably a human bodily fluid like serum, plasma, or blood, depending on the test being performed. Further, the test card (214) comprises a quality control marker (215), at least two detection lines (216, 217), and a quality control line (218).

The at least two detection lines (216, 217) may have at least one IgG line (216), and at least one IgM line (217). Tests are understood by understanding the visibility and intensity of the at least two detection lines (216, 217), and the quality control line (218).

At step 150, the user uploads a picture or scan of the testing kit (210) once the test has been performed. The picture or scan may be captured by using a scanner (263) provided in the user module (260). The user module (260) further comprises a user processing unit (261), a central server (262), and a network module (264). Once the picture or scan has been captured appropriately, the user utilises the user interface (265) to upload the picture or scan to an examination module (240)

At step 160, the picture or scan of the testing kit (210) is sent to the examination module (240) for the analysis of a diagnosis based on the picture or scan of the testing kit (210). The examination module comprises a deep learning module (241) and a learning database (243). Further, the deep learning module (241) comprises a bounding region calculator (242), an image recognition module (245), and an intensity unit (244). The examination module (240) utilises the bounding region calculator (242) to bound the region of the picture or scan of the testing kit (210) where the test card (214) is located.

The image recognition module (245) recognises the at least two detection lines (216, 217) and the quality control line (218). Once the lines have been appropriately recognised as the at least one IgG detection line (216), the at least one IgM detection line (217), and the quality control line (218), the image recognition module (245) notes the visibility and intensity of the lines.

The result of the test depend on the at least one IgG detection line (216), the at least one IgM detection line (217), and the quality control line (218). Regardless of the result, the quality control line (218) should be visible. If the quality control line (218) is not visible, that means that the test performed is invalid, and that another test should be performed again.

When the at least two detection lines (216, 217) including the at least one IgG detection line (216), and the at least one IgM detection line (217) are visible along with the quality control line (218), it means that the user is testing positive for the condition being diagnosed. However, when there is no colour in in the at least two detection lines (216, 217) including the at least one IgG detection line (216), and the at least one IgM detection line (217), meaning when these lines are not visible, it is considered that the user is testing negative for the condition being diagnosed.

Further, the intensity unit (244) identifies the intensity of colour of the quality control line (218). The intensity of colour in the quality control line (218) provides an estimate of the degree to which a condition has reached. If a positive diagnosis is attained, then it is facilitated by the estimate of the degree to which a condition has reached in the diagnosis as well.

At step 170, the diagnosis is sent to a medical certificate module (250) by the examination module (240). The medical certificate module (250), at step 180, issues a medical certificate to the user based on the diagnosis provided by the examination module (240).

The medical certificate module (250) comprises of a certificate database (251), a blockchain node (252), a blockchain server (253) and a certificate processing unit (254). The medical certificate is issued for a user only if the identity of the user was successfully authenticated vis-à-vis the identity database (234). The certificate processing unit (254) creates the medical certificate and stores it on the blockchain node (252), while also saving the medical certificate as well as the blockchain node (252) on the certificate database (251). From the blockchain node (252), the medical certificate is sent to the blockchain server (253), which forwards the medical certificate to the user interface (265), from where the user can view or download the medical certificate.

The issued medical certificates are also provided with unique identification codes which can be scanned in order to verify and authenticate the medical certificates. If a wrongful medical certificate is scanned, it alerts the authorities to forgery of the medical certificate.

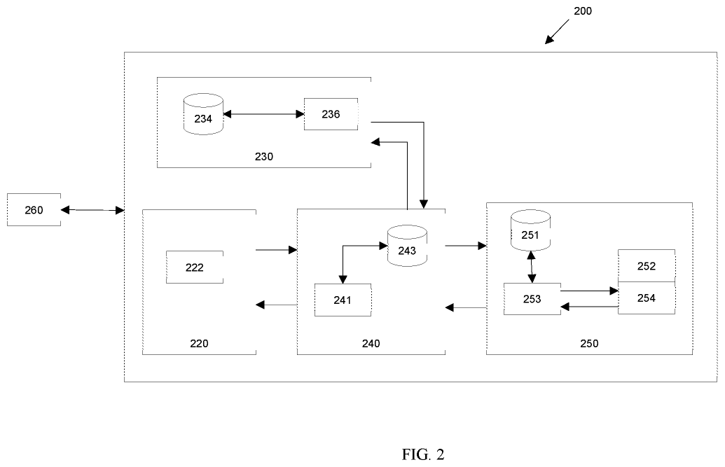

Referring to FIG. 2 illustrates a block diagram depicting the system for authentication and examination of test samples (200), in accordance with an embodiment of the present disclosure. As mentioned in FIG. 2, the system for authentication and examination of test samples (200) comprises of the identity authentication module (230), the examination module (240), the kit authentication module (220), and the medical certificate module (250); and is communicably coupled to the user module (260).

The identity authentication module (230) the system for authentication and examination of test samples (200) comprises the identity module (236) and the identity database (234) communicably coupled together. The examination module (240), comprises the deep learning module (241) and the learning database (243) communicably coupled together. Additionally, the examination module (240) is connected to the identity authentication module (230).

Further, the kit authentication module (220) comprises a plurality of coded information (222) and is connected to the examination module (240). The medical certificate module (250) comprises a certificate database (251), a blockchain node (252), a blockchain server (253), and a certificate processing unit (254), all communicably coupled together Furthermore, the medical certificate module (250) is connected to the examination module (240).

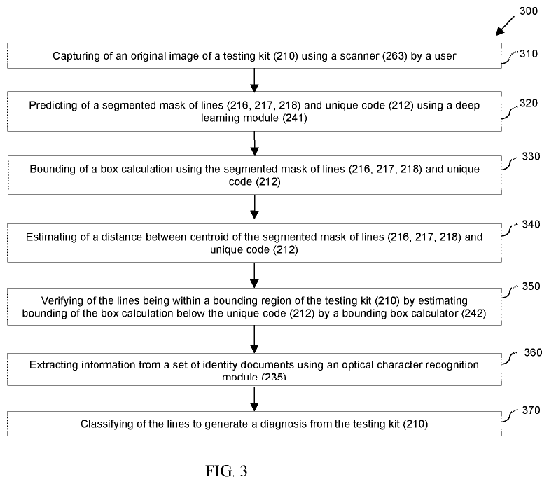

Referring to FIG. 3 illustrates a process flow diagram depicting a method for inputting of information (300) in the system for authentication and examination of test samples (200), in accordance with an embodiment of the present disclosure. Furthermore, the method for inputting of information (300) comprises of multiple steps as recited below.

At step 310, the user captures an original image of the testing kit (210) using the scanner (263) comprised in the user module (260). The original image of the testing kit is provided to the examination module (240).

At step 320, the deep learning module (241) of the examination module (240) predicts a segmented mask of lines (216, 217, and 218) and unique code (212). At step 330, the bounding box calculator (242) of the deep learning module (241) bounds a box calculation using the segmented mask of lines (216, 217, and 218) and unique code (212).

At step 340, the bounding box calculator (242) estimates a distance between centroid of the segmented mask of lines (216, 217, and 218) and unique code (212). At step 350, the bounding box calculator (242) verifies the lines being within a bounding region of the testing kit (210) by estimating bounding of the box calculation as being placed below the unique code (212).

At step 360, the optical recognition module (235) comprised in the identity authentication module (230) extracts information from a set of identity documents along with the document module (231) and verifies the same with the identity database (234) in order to authenticate the identity of the user.

At step 370, the examination module (240), and specifically the image recognition module (245) of the examination module (240) classifies the lines (216, 217, and 218) in order to generate a diagnosis from the testing kit (210).

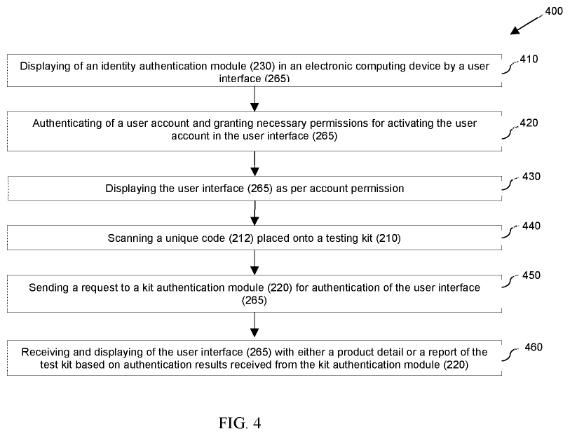

Referring to FIG. 4 illustrates a process flow diagram depicting a method for authentication of test kits (400) by the system for authentication and examination of test samples (200), in accordance with an embodiment of the present disclosure. Furthermore, the method for authentication of test kits (400) comprises of multiple steps as recited below.

At step 410, the user interface (265) displays identity authentication module (230) in an electronic computing device. At step 420, identity authentication module (230) authenticates a user account and grants necessary permissions for activating the user account in the user interface (265).

At step 430, the user interface (265) is displayed as per permissions provided to the user account. At step 440, the unique code (212) placed onto the kit body (211) of the testing kit (210) is scanned by the user using a scanner (263) of the user module (260).

At step 450, the user interface (265) sends a request to the kit authentication module (220) for authentication of the testing kit (210). At step 460, the user interface (260) receives and displays either a product detail or a report of the testing kit (210) based on authentication results received from the kit authentication module (220).

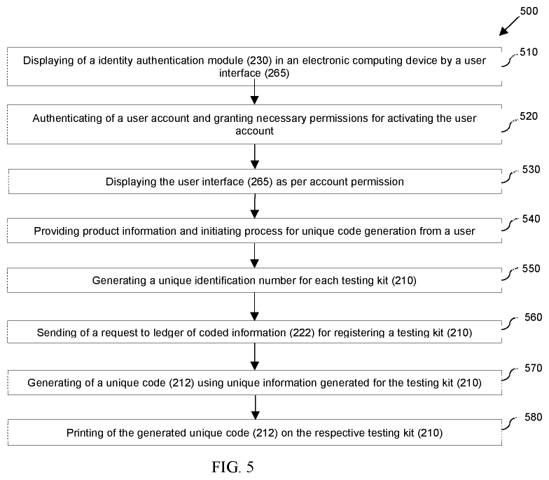

Referring to FIG. 5 illustrates a process flow diagram depicting a method for registration of test kits (500) by the system for authentication and examination of test samples (200), in accordance with an embodiment of the present disclosure. Furthermore, the method for registration of test kits (500) comprises of multiple steps as recited below.

At step 510, the user interface (260) displays the identity authentication module (230) in the electronic computing device. Once the identity of the user is confirmed, the identity authentication module (230), at step 520, authenticates the user account and grants necessary permissions for activating the user account.

At step 530, the user interface (260) is displayed as per account permissions granted. At step 540, the user provides product information and initiates a process for unique code generation. At step 550, the kit authentication module (220) generates a unique identification number for each testing kit (210).

At step 560, the kit authentication module (220) sends a request to the ledger of coded information (222) for registering a testing kit (210). At step 570, the kit authentication module (220) generates the unique code (212) using the unique identification number information generated for the testing kit (210). And at step 580, during the manufacturing process, the generated unique code (212) is printed onto the respective testing kit (210).

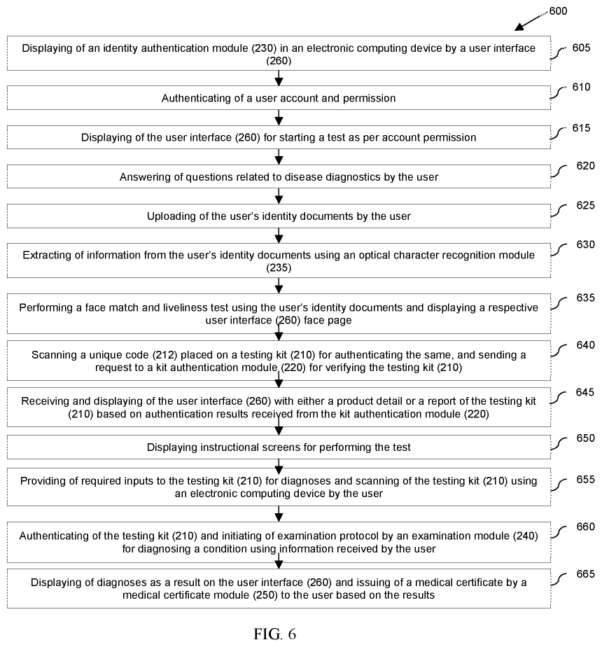

Referring to FIG. 6 illustrates a process flow diagram depicting a method for authentication and examination of test samples (600), in accordance with an embodiment of the present disclosure. Furthermore, the method for authentication and examination of test samples (600) comprises of multiple steps as recited below.

At step 605, the user interface (260) displays the identity authentication module (230) in the electronic computing device. At step 610, the user account is authenticated and grated necessary permissions. At step 615, the user interface (260) is displayed for starting a test as per account permissions granted.

At step 620, the user answers questions related to disease diagnostics displayed on the user interface (260). At step 625, the user uploads identity documents onto the user interface (260). At step 630, the optical character recognition module (235) extracts information from the user's identity documents along with the document module (231).

At step 635, the identity authentication module (230) performs the face match test (232) and the liveliness test (233) using the user's identity documents and displays a respective face page on the user interface (260). At step 640, the user scans the unique code (212) placed on the testing kit (210) for authenticating the same, and in turn, the user module (260) sends a request to the kit authentication module (220) for verifying the testing kit (210).

At step 645, the user interface (260) receives authentication results from the kit authentication module (220), and displays on the user interface (260) either a product detail or a report of the testing kit (210) based on authentication results received.

At step 650, the user interface (260) displays instructional screens for performing the selected test. At step 655, the user provides required inputs to the testing kit (210) for diagnoses and scanning of the testing kit (210) using the electronic computing device.

At step 660, the test kit authentication module (220) authenticates the testing kit (210) based on the information received from the user, and initiates examination protocol by an examination module (240) for diagnosing a condition. At step 665, the examination module (240) displays the diagnosis as a result on the user interface (260) and issues the medical certificate via the medical certificate module (250) to the user based on the results.

In an aspect of the present embodiment, the system for authentication and examination of test samples (200) and the method for authentication and examination of test samples (100) may also comprise of a marketplace where manufacturers, consumers and suppliers can connect for commercial dealings in order to ensure distribution of authenticated testing kits (210) in the market.

In another aspect of the present embodiment, the system for authentication and examination of test samples (200) and the method for authentication and examination of test samples (100) may also comprise of a wallet module facilitated in the user module (260) as an online service that allows users to make electronic transactions with other parties, bartering digital units for goods and services. Herein, the wallet module may include transactions of digital currency or nationally recognised currencies, as per the exchange rate valid at that point of time.

In yet another aspect of the present embodiment, the system for authentication and examination of test samples (200) and the method for authentication and examination of test samples (100) may also comprise of a catalogue management feature which enables medical products to be made available to interested organisations or consumers in order to facilitate procuring of electronic goods, as well as hosting medical products on their platform

As will be readily apparent to a person skilled in the art, the present invention may easily be produced in other specific forms without departing from its essential composition and properties. The present embodiments should be construed as merely illustrative and non-restrictive and the scope of the present invention being indicated by the claims rather than the foregoing description, and all changes which come within therefore intended to be embraced therein.

I/We claim:

1. A method for authentication and examination of test samples (100), comprising the steps of
availing a testing kit (210) by a user;
authenticating the user's identity via a user module (260), communicably coupled with an identity authentication module (230);
authenticating the testing kit (210) by the user via a kit authentication module (220);
performing a test by the user utilising the testing kit (210);
uploading a picture or scan of the testing kit (210) by the user on an examination module (240) via the user module (260), wherein the picture or scan of the testing kit (210) is uploaded once the test has been performed;
analysing of a diagnosis based on the picture or scan of the testing kit (210) by the examination module (240);
sending of the diagnosis to a medical certificate module (250) by the examination module (240); and
issuing of a medical certificate based on the diagnosis by the medical certificate module.

2. The method for authentication and examination of test samples (100) as claimed in claim 1, wherein the user's identity is authenticated by the identity authentication module (230) by way of a set of identity documents, a face match test (232), a liveliness test (233), or more than one of the set of identity documents, the face match test (232), and the liveliness test (233).

3. The method for authentication and examination of test samples (100) as claimed in claim 2, wherein the identity authentication module (230) reads the set of identity documents via an optical character recognition module (235).

4. The method for authentication and examination of test samples (100) as claimed in claim 1, wherein the testing kit (210) has a kit body (211) provided with a unique code (212).

5. The method for authentication and examination of test samples (100) as claimed in claim 4, wherein the testing kit (210) is authenticated by the kit authentication module (220) by reading the unique code (212) placed on the kit body (211) via a code reader (221) and verifying it with a ledger of encoded information (222).

6. The method for authentication and examination of test samples (100) as claimed in claim 1, wherein the examination module (240) analyses a diagnosis based on the picture or scan of the testing kit (210) by way of a deep learning module (241) and a learning database (243) comprised in the examination module (240).

7. A system for authentication and examination of test samples (200), comprising
an identity authentication module (230), having an identity module (236) and an identity database (234) communicably coupled together;
an examination module (240), having a deep learning module (241) and a learning database (243) communicably coupled together, wherein the examination module (240) is connected to the identity authentication module (230);
a kit authentication module (220), having a plurality of coded information (222), wherein the kit authentication module (220) is connected to the examination module (240); and
a medical certificate module (250), having a certificate database (251), a blockchain node (252), a blockchain server (253), and a certificate processing unit (254) communicably coupled together, wherein the medical certificate module (250) is connected to the examination module (240);
wherein the system for authentication and examination of test samples (200) is communicably coupled to a user module (260).

8. The system for authentication and examination of test samples (200) as claimed in claim 7, wherein the system for authentication and examination of test samples (200) is utilized for authenticating a testing kit (210), the testing kit (210) comprising
a kit body (211), having
a unique code (212) provided on the kit body (211);
a sample storage area (213) wherein a bodily fluid sample is placed; and
a test card (214) having a quality control marker (215), at least two detection lines (216, 217), and a quality control line (218).

9. The system for authentication and examination of test samples (200) as claimed in claim 8, wherein the testing kit (210) is authenticated by the kit authentication module (220) via a code reader (221) provided in the kit authentication module (220) which reads the unique code (212) placed onto the kit body (211), and verifies the unique code (212) with the plurality of coded information (222).

10. The system for authentication and examination of test samples (200) as claimed in claim 8, wherein the deep learning module (241) comprises
a bounding region calculator (242) which bounds the region of the test card (214) having a performed test result;
an image recognition module (245) which recognizes the quality control marker (215), the at least two detection lines (216, 217), and the quality control line (218); and
an intensity unit (244) which identifies the intensity of the detection lines (216, 217), and the quality control line (218) in order to calculate a degree to which a condition has reached, and ultimately providing a diagnosis.
